(19) United States
(12) Patent Application Publication
(10) Pub. No.: US 2019/0343709 A1
(43) Pub. Date: Nov. 14, 2019

(54) Wardlow Seat Strap

(71) Applicant: Baari Wardlow, Belmont, CA (US)

(72) Inventor: Baari Wardlow, Belmont, CA (US)

(21) Appl. No.: 15/993,672

(22) Filed: May 31, 2018

Related U.S. Application Data
(60) Provisional application No. 62/670,437, filed on May 11, 2018.

(51) Int. Cl. A61H 3/00; A61G 5/10; A61F 5/37

(52) U.S. Cl. CPC A61H 3/00; A61G 5/1091; A61H 2201/0161; A61H 2201/1633; A61F 5/3792

(57) ABSTRACT

The Wardlow Seat Strap is a seat that attaches to any standard hospital walker and gives the user a place to sit down as needed to reduce the risk of falling while using the walker.

### BACKGROUND & SUMMARY

It is a state-of-the-art Seat Strap that is affixed to the handles of a hospital walker and gives the patient a convenient and secure place to sit down at any time while using the walker.
Additionally, it is a seat that is attached to the hospital walker which is adjustable and light enough to transport.

### DESCRIPTION OF DRAWINGS

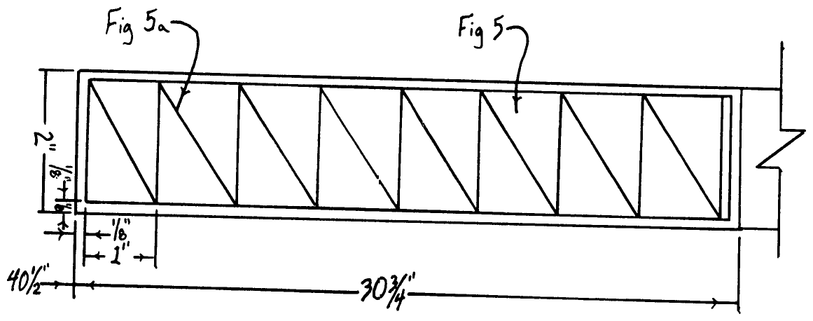

Page 1-10 is an exploded view of the Strap with the folded down front end which holds the two D-rings and the heavy-duty hook Velcro. This Velcro attaches the strap to the left handle of the walker when pressed against it.
Page 2-10 is a view of the Strap showing both sides in its entirety and complete.
Page 3-10 is a view of the 18-inch hook Velcro that secures the Strap to the right handle of the walker.
Page 4-10 is a view of the loop Velcro that is glued to both left and right handles of the walker for security.
Page 5-10 is an exploded view of the hook Velcro sewn to side B. This Velcro secures the end of the Strap.
Page 6-10 is an exploded view of the loop Velcro sewn to side B. This Velcro gives the security to the Strap when pressed against the hook Velcro, as shown on FIG. 5.
Page 7-10 is a view of the top and the bottom of the Seat.
Page 8-10 is a view of the first drawing made of the Wardlow Seat Strap.
Page 9-10 is a three-dimensional view of the Seat, the loop Velcro, and where it is sewn.
Page 10-10 is a view of the Wardlow Seat Strap as it appears when attached to the hospital walker while secured with Velcro to allow walking.

### DETAILED DESCRIPTION OF THE INVENTION

The Wardlow Seat Strap is a 2″ wide, 142½″ long nylon strap which has a 5⅝″ by 2″ hook Velcro on side A staring at 2¾″ from a fold holding two D-rings. The second hook Velcro is 18″ by 2″ starting 45½″ from the fold holding the D-ring, and on side B, a 30¾″ by 2″ piece of hook Velcro starting at 40½″ from the D-ring fold and sewn down. The 57¾″ piece of loop Velcro, on side B, starts at 85″ from the D-ring fold sewn down. The 5⅝″ hook Velcro on side A is pressed against the 4½″ by 2″ loop Velcro glued onto the left handle of the walker. The 18″ by 2″ hook Velcro is pressed against the 4½″ by 2″ loop Velcro, glued onto the right handle of the walker, then being fed through the belt loops on the bottom of the Seat. The Seat has a 6″ by 2″ piece of loop Velcro that is pressed against the 3⅝″ by 2″ piece of hook Velcro centered and glued onto the crossbar of the walker. The Strap is buckled with the two 2″ D-rings, and the excess length of strap with the 57¾″ by 2″ loop Velcro on side B is pressed against the 30¾″ by 2″ hook Velcro, for added security against slippage
The Seat can be adjusted to fit the height of each individual patient as appropriate. It has the strength to hod individuals up to 300 pounds, as long as their hips fit within the walker handles.

### SUMMARY OF THE INVENTION

Accordingly, it is a seat that is attached to the hospital walker for the convenience of sitting down at any time while using the walker.

### BRIEF DESCRIPTION OF THE DRAWINGS

For a fuller understanding of the nature and objects of the invention, reference should be made to the following detailed descriptions taken in conjunction with the accompanying drawings, in which:

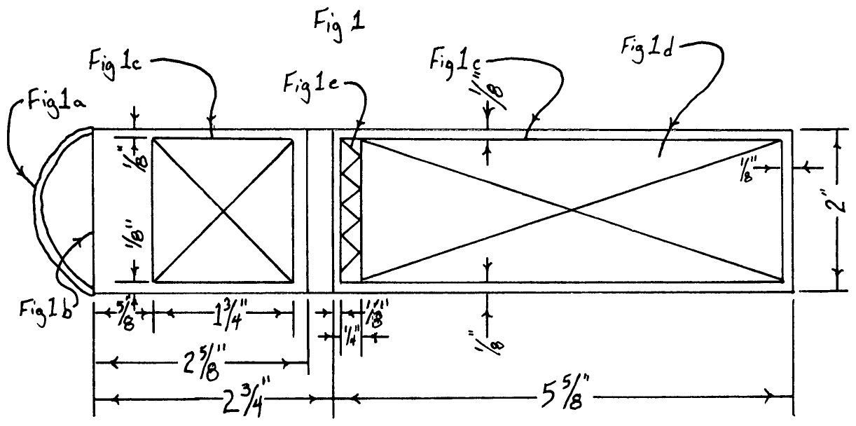

FIG. 1 is a pictorial exploded view showing the first end of the Strap, Side A, that secures the D-rings by folding the Strap at a length of 2⅝″ and using double-stitching. Continuing down the Strap 2¾″ is a 5⅝″ by 2″ piece of hook Velcro with double-stitching that is used to secure the Strap to the handle of the walker.

FIG. 1a is a pictorial view of the two 2″ D-rings.

FIG. 1b is a pictorial view of the 2″ strap folded down 2⅝″.

FIG. 1c is a pictorial view of the double-stitching with heavy-duty thread.

FIG. 1d is a pictorial view of the 5⅝″ by 2″ piece of heavy-duty hook Velcro.

FIG. 1e is a pictorial view of the zigzag stitching at ¼″ to give added security to the end of the hook Velcro.

FIG. 1f is a pictorial view of the double-stitching with heavy-duty thread spaced 1″ apart, with a diagonal stitch from top left to bottom right.

FIG. 1g is a pictorial view of the heavy-duty hook Velcro measuring 18 by 2″.

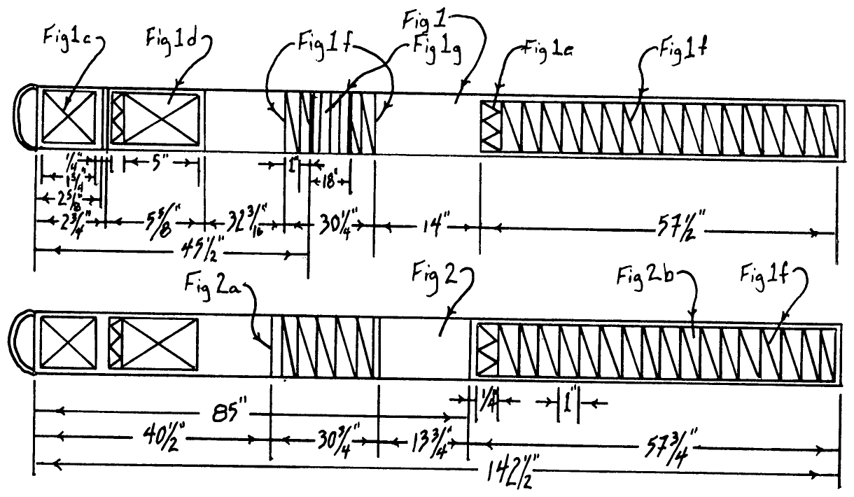

FIG. 2 is a pictorial view of Side B of the 142½″ heavy-duty nylon Strap with two 2″ D-rings attached by a 2⅝″ fold at the beginning of the Strap, which is sewn down.

FIG. 2a is a pictorial view of the 30¾″ by 2″ heavy-duty hook Velcro.

FIG. 2b is a pictorial view of the 57¾″ by 2″ heavy-duty loop Velcro.

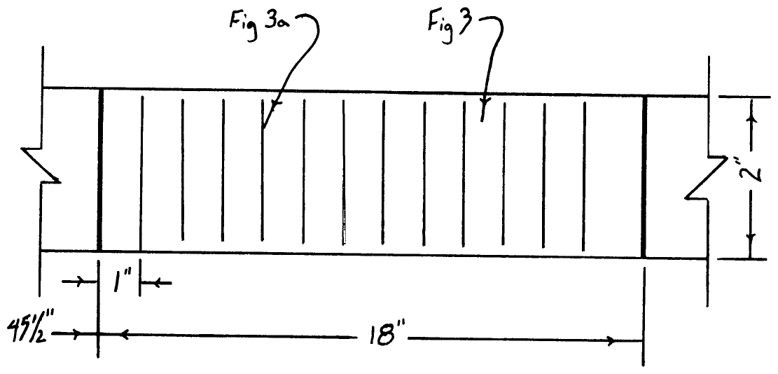

FIG. 3 is a pictorial exploded view of the heavy-duty hook Velcro (as referenced in FIG. 1g).

This hook Velcro is for the right handle of the walker and, when pressed against the 4½″ by 2″ loop Velcro which is glued to the handle of the walker, will give added security against slippage, and can be adjusted to the desired height. This hook Velcro attached to Side A starts at 45½″ from the D-ring fold.

FIG. 3a is a pictorial exploded view of the 1″ wide parallel stitching with heavy duty thread, which is continuous through the 13″ hook Velcro.

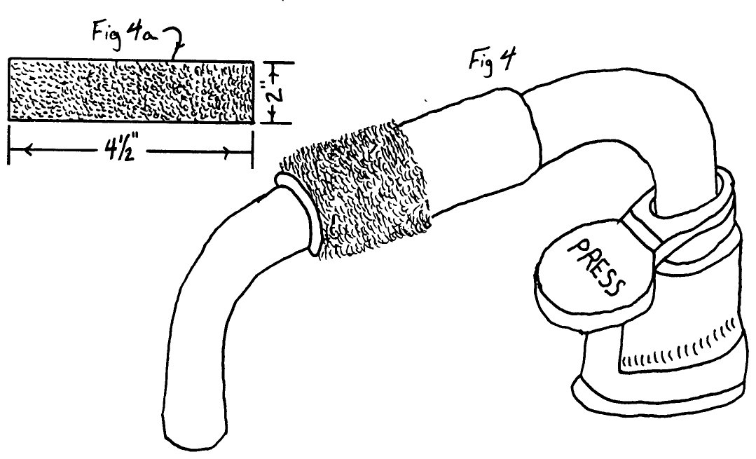

FIG. 4 is a pictorial exploded view of the left and/or right handles of the hospital walker A 4½″ by 2″ piece of heavy-duty loop Velcro is glued to the soft handle on the end, away from the “Press” lever (as referenced in

FIG. 4a).

FIG. 4a is a pictorial view of the 4½″ by 2″ heavy-duty loop Velcro.

FIG. 5 is a pictorial exploded view of the 30¾″ by 2″ hook Velcro sewn to Side B, starting at 42½″ from the fold holding the two 2″ D-rings.

FIG. 5a is a pictorial view of the ⅛″ edge to edge heavy-duty thread stitching with 1″ spacing, which is continuous through the 30¾″ hook Velcro.

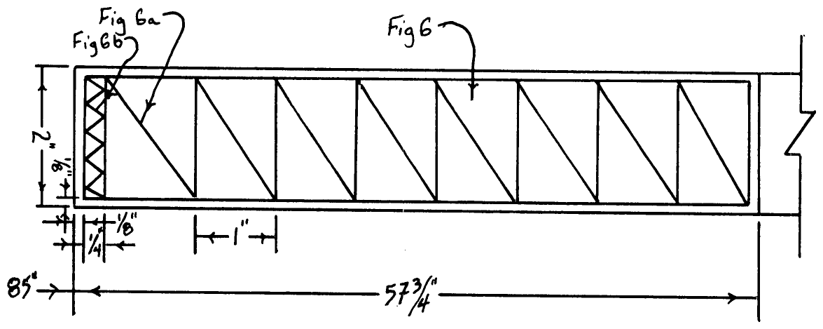

FIG. 6 is a pictorial exploded view of the 57¾″ by 2″ heavy-duty loop Velcro sewn to Side B of the Strap, starting 85″ from the fold holding the two 2″ D-rings

FIG. 6a is a pictorial view of the ⅛″ edge to edge heavy-duty thread stitching with 1″ spacing, which is continuous through the 57¾″ loop Velcro.

FIG. 6b is a pictorial view of the ¼″ zigzag stitch.

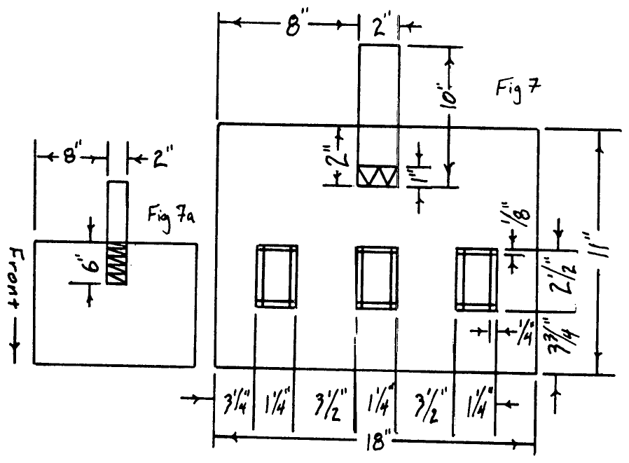

FIG. 7 is a pictorial view of the bottom of the 18″ by 11″ seat, showing the three 2½″ by 1¼″ belt loops, starting from the front left corner, going 3¼″ towards the right and 3¾″ towards the back. The 10″ by 2″ piece of heavy-duty hook Velcro is sewn with a 1″ by 2″ zigzag stitch with heavy-duty thread, starting 2″ from the back and 8″ from the side.

FIG. 7a is a pictorial perspective view of the 18″ by 11″ seat showing the 6″ by 2″ piece of loop Velcro sewn to the seat with 1″ spacing, which is continuous through the 6″ piece of heavy-duty loop Velcro. The 6″ by 2″ piece of heavy-duty loop Velcro has a cross-stitch from top left to bottom right, starting from the back of the seat and 8″ from the side.

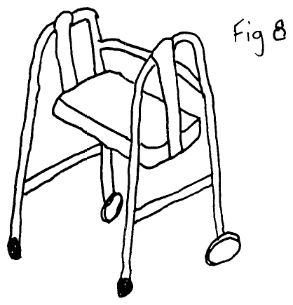

FIG. 8 is a pictorial view showing the first drawing of the Wardlow Seat Strap.

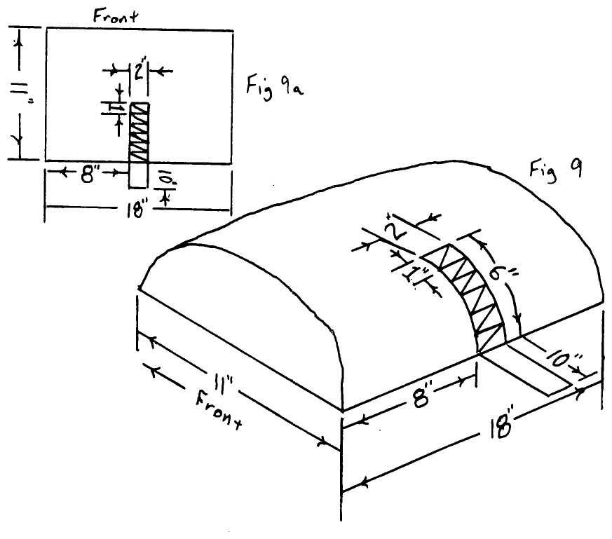

FIG. 9 is a pictorial exploded view of the 18″ by 11″ seat, showing the 6″ by 2″ heavy-duty loop Velcro sewn over the top, and the 10″ by 1″ heavy-duty hook Velcro sewn from underneath.

FIG. 9a is a pictorial view of the top of the 18″ by 11″ seat, showing the 6″ by 2″ heavy-duty hook Velcro, sewn with 1″ stitches of heavy-duty thread, and a cross-stitch going from top left to bottom right corner of each 1″ stitch.

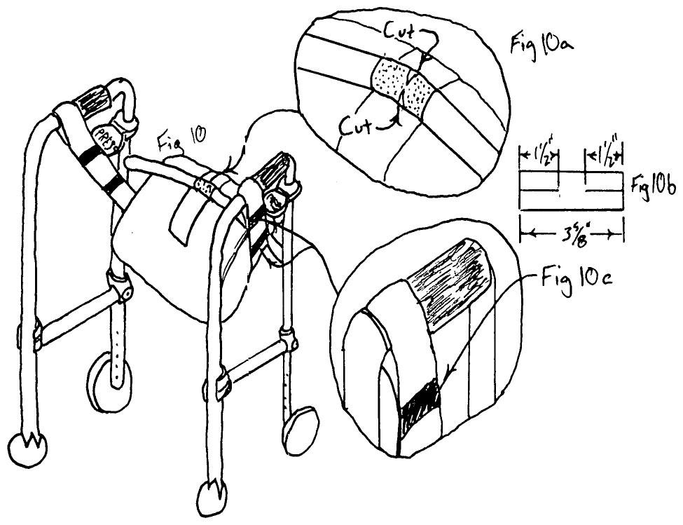

FIG. 10 is a pictorial view of the Seat Strap attached to the walker, and the 18″ by 11″ seat attached to the hook and loop Velcro on the front cross bar of the walker.

FIG. 10a is a pictorial exploded view of the 3⅝″ by 2″ heavy-duty hook Velcro glued around the center of the front cross bar of the walker.

FIG. 10b is a pictorial view of the cuts on the heavy-duty hook Velcro 1½″ from the left and 1½″ from the right (as referenced in FIG. 10a)

FIG. 10c is a pictorial view of one of the four hook and loop Velcro fasteners on the top and bottom of the Strap, next to the handles of the walker and the seat. These fasteners are placed to give additional support and prevent slippage of the Seat.

What is claimed is:

1. The Wardlow Seat Strap is a one-piece nylon heavy duty 2″ strap with heavy duty 2″ Velcro sewn on that is attached to an 18″ by 11″ padded seat and pressed against the handles of the hospital walker, which allows the patient to sit down anywhere at any time.

2. The one-piece strap of claim 1 is constructed from 2″ wide nylon, which is 142½″ long and ⅛″ thick.

3. The hook and loop Velcro of claim 1 is heavy duty, 2″ wide, and can hold anyone up to 300 lbs. who can fit between the handles of the walker.
